(19) United States
(12) Patent Application Publication
(10) Pub. No.: US 2003/0144596 A1
(43) Pub. Date: Jul. 31, 2003

(54) Strap structure and a biological information sensing device using the same

(76) Inventor: Keisuke Tsubata, Chiba-shi (JP)

Correspondence Address: ADAMS & WILKS, 31st Floor, 50 Broadway, New York, NY, 10004, US

(21) Appl. No.: 10/349,882

(22) Filed: Jan. 23, 2003

(30) Foreign Application Priority Data
Jan. 31, 2002 (JP) 2002-022823

(51) Int. Cl. A61B 5/02

(52) U.S. Cl. 600/500

(57) ABSTRACT

A strap structure having a strap wrapped around a neck-like portion in a predetermined fastened state and a biological information sensing device using the same are provided. A strap structure adapted to apply a main body of an arterial pulse wave detector or the like onto a wrist portion or the like is designed to be worn on the neck-like portion with its strap body wrapped around the neck-like portion. The strap body includes an elastic strap-like portion elastically elongated according to a tension applied to the strap body, and elongation indicating means, M for indicating an elongation of the elastic strap-like portion.

### BACKGROUND OF THE INVENTION

1. Field of the Invention

The present invention relates to a strap structure suitable for use with a biological information sensing device for sensing biological information such as arterial pulses or the like, as well as to a biological information sensing device using the same or more particularly, a biological information sensing device of a type like an arterial pulse wave detector which is wrapped around the wrist or the like.

2. Description of the Related Art

A portable arterial pulse wave detector has been proposed which is provided with a pressure forming elastic piece at an intermediate portion of a strap in a manner to permit adjustment for its position along an extending direction of the strap, such that the elastic piece may press a sensor portion against an area of the wrist surface near the radial artery of the wrist thereby holding the sensor portion in intimate contact with the wrist surface area (Japanese Unexamined Patent Publication No. 8(1996)-52118).

However, the proposed portable arterial pulse wave detector has a drawback that precise measurements of arterial pulses cannot be obtained. This is because the detector is fastened about the wrist by pressing the sensor portion and the pressure forming elastic piece against two places on the wrist so that it is difficult to set a desired pressure at which the sensor portion is pressed against a measurement area.

In view of the foregoing, it is an object of the invention to provide a strap structure for permitting a strap to be wrapped around a neck-like portion in a predetermined fastened state and a biological information sensing device using the same.

### SUMMARY OF THE INVENTION

In accordance with the invention for achieving the above object, a strap structure applied to a neck-like portion with its strap body wrapped around the neck-like portion, the strap structure is characterized in that the strap body includes an elastic strap-like portion elastically elongated according to a tension applied to the strap body, and elongation indicating means for indicating an elongation of the elastic strap-like portion.

According to the strap structure of the invention, the strap body includes “the elastic strap-like portion elastically elongated according to a tension applied to the strap body and besides, the elongation indicating means for indicating the elongation of the elastic strap-like portion”. Therefore, it is possible to stretchingly wrap the strap body around the neck-like portion as visually checking the indication of the indicating means, so as to restrict the elongation indicated by the elongation indicating means within a predetermined range. This permits a fastening force (tension) for fastening the strap body about the neck-like portion to be positively set to a desired magnitude. It is noted here that the elongation indicating means can act as a fastening force (indicating) gauge.

The elastic strap-like portion or stretchable strap-like portion is typically disposed at one place with respect to a longitudinal direction of the strap body. If desired, however, the elastic strap-like portion may be provided at plural places. Otherwise, there may be a case where the elastic strap-like portion substantially extends the overall length of the strap body.

The strap structure according to the invention is characterized in that the elongation indicating means typically includes an indicator frame having one end thereof attached to a non-elastic (non-stretchable) strap-like portion connected with one end of the elastic strap-like portion and the other end thereof extended along an extension direction of the elastic strap-like portion; and an elongation indicating mark attached to a portion of the strap body that corresponds to the extended end of the indicator frame and is designed to be exposed from the indicator frame. In this case, the elongation may be comprehended from the amount of displacement of the elongation indicating mark relative to the indicator frame. So long as positive visual recognition is allowed, the mark may comprise a character, line, pattern, color gradations or color variations, or may take other indication modes.

The indicator frame typically acts as a cover for hiding the elongation indicating mark located within the frame. If desired, the indicator frame may be provided for the view of the mark located within the frame and may be marked with an arrow or scale such that the indication by way of the mark may be comprehended based on the arrow or scale.

The indicator frame is typically formed in a flat tubular shape, through which a part of the strap body is inserted. However, the indicator frame may have other shapes such as one having a U-shaped horizontal section, a flat plate-like shape and the like.

The strap structure according to the invention further comprises an elongation restricting member for defining an upper limit of the elongation of the elastic strap-like portion. This is effective to prevent the elastic strap-like portion from being elongated so much to be broken or being deteriorated too quickly. The elongation restricting member typically comprises a separate non-stretchable member in the form of a strap or the like, opposite ends of which are connected with non-elastic strap-like portions connected with opposite ends of the elastic strap-like portion.

In the strap structure according to the invention, the amount of elongation is practically corresponds to the tension applied to the strap structure and hence, the strap structure of the invention may be defined in terms of tension. Thus, a strap structure according to the invention for achieving the above object which is applied to a neck-like portion with its strap body wrapped around the neck-like portion is characterized in that the strap body includes an elastic strap-like portion elastically elongated according to a tension applied to the strap body, and a tension indicating means for indicating a tension applied to the elastic strap-like portion.

In this case, the strap structure according to the invention is characterized in that the tension indicating means typically includes an indicator frame having one end thereof attached to a non-elastic strap-like portion connected with one end of the elastic strap-like portion and the other end thereof extended along an extension direction of the elastic strap-like portion; and a tension indicating mark attached to a portion of the strap body that corresponds to the extended end of the indicator frame and is designed to be exposed from the indicator frame. Typically, the strap structure according to the invention further comprises tension restricting member for defining an upper limit of the tension applied to the elastic strap-like portion.

In either case where the amount of elongation is indicated or where the amount of tension is indicated, the strap structure according to the invention typically comprises a fastening hardware provided at one end of the strap body and including an aperture in which the other end of the strap body is inserted; and fixing means for fixing the other end portion of the strap body inserted through the aperture of the fastening hardware in a manner to allow for the definition of an optional length of the inserted other end portion.

It is noted here that the ability to define an optional length of the inserted end portion of the strap body corresponds to the ability to stretch or fasten the strap body into a desired elongation or tense state. This facilitates a positive application of the strap body onto the neck-like portion in a desired elongation or tense state. The aperture of the fastening hardware may have its entire periphery enclosed or may be formed with a notch at a portion thereof. It is noted that the strap body may be fastened in any other fashion.

In accordance with the invention for achieving the above object, a biological information sensing device worn on a neck-like portion, inclusive of a predetermined measurement area, of a living organism as holding a sensor portion thereof in intimate contact with the measurement area for sensing biological information of the living organism, the sensing device comprises a main body including the sensor portion and a flexible strap-like sensor holder for holding the sensor portion; and the strap structure having the aforementioned construction to fasten the main body of the biological information sensing device to the neck-like portion of the living organism.

The biological information sensing device is adapted to apply the sensor portion of the main body of the sensing device to the neck-like portion at a desired pressure in an easy and positive manner. Hence, the sensor portion can take precise measurements of the biological information. Furthermore, even when the neck-like portion is moved during exercise or the like, the main body of the sensing device can readily and positively sense the biological information. It is noted that the strap structure may also be used for fastening any article other than the main body of biological information sensing device to the neck-like portion.

The biological information sensing device according to the invention is characterized in that the sensor holder of the main body of the biological information sensing device supports, in addition to the sensor portion, an actuating portion for the sensor portion and a circuit portion including a processing portion for processing the biological information sensed by the sensor portion. However, the sensor portion alone may be applied to the neck-like portion. The sensor portion may comprise, for example, a sensor for sensing information on blood flowing through the radial artery. However, the sensor portion may comprise any other sensor so long as it is desirably pressed against the measurement area at a desired pressure.

### BRIEF DESCRIPTION OF THE DRAWINGS

FIG. 1 are groups of diagrams showing a strap structure according to one preferred embodiment of the invention, FIGS. 1A and 1B representing plan views explanatory of a strap structure in development (in flattened out position), FIGS. 1C and 1D representing side views explanatory of the strap structure, and FIGS. 1E and 1F representing side views explanatory of a strap body of the strap structure (including elongation restricting means) from which a tubular portion is removed, FIGS. 1A, 1C and 1E each showing the strap body free from tension (or a tension is less than an optimum level), whereas FIGS. 1B, 1D and 1F each showing the strap body under a tension of the optimum level (magnitude);

FIG. 2 are groups of diagrams showing an arterial pulse wave detector according to one preferred embodiment of the invention which is formed by assembling a main body of an arterial pulse wave detector with the strap structure of FIG. 1 and which is applied to the wrist, FIG. 2A representing a side view (or a horizontal sectional view of the wrist) explanatory of a state where the arterial pulse wave detector is applied, FIGS. 2B and 2C representing views as seen along the arrow II in FIG. 2A, FIG. 2B showing a state where a fastening force is yet to reach the optimum level whereas FIG. 2C showing a state where the fastening force is at the optimum level;

FIG. 3 are groups of diagrams specifically illustrating an example of a structure and state of a tension indicator portion including the tubular portion when the tension on the strap structure of FIG. 1 is less than the optimum level, FIG. 3A representing a sectional view taken on the line IIIA-IIIA in FIG. 3B, FIG. 3B representing a sectional view taken on the line IIIB -IIIB in FIG. 3A, and FIG. 3C representing a sectional view taken on the line IIIC-IIIC in FIG. 3B;

FIG. 4 are groups of diagrams specifically illustrating an example of the structure and state of the tension indicator portion including the tubular portion when the tension on the strap structure of FIG. 1 is at the optimum level, FIG. 4A representing a sectional view taken on the line IVA-IVA in FIG. 4B, FIG. 4B representing a sectional view taken on the line IVB-IVB in FIG. 4A, and FIG. 4C representing a sectional view taken on the line IVC-IVC in FIG. 4B;

FIG. 5 are groups of diagrams specifically illustrating an example of the structure and state of the tension indicator portion including the tubular portion when the tension on the strap structure of FIG. 1 is much more greater than the optimum level, FIG. 5A representing a sectional view taken on the line VA-VA in FIG. 5B, and FIG. 5B representing a sectional view taken on the line VB-VB in FIG. 5A;

FIG. 6 are groups of diagrams illustrating an exemplary modification of the tension indicator portion of the strap structure as a modification of the strap structure of the invention, FIGS. 6A to 6C representing similar sectional views to FIG. 3A, respectively showing a state where the tension is less than the optimum level, a state where the tension is at the optimum level, and a state where the tension is much more greater than the optimum level;

FIG. 7 are groups of functional block diagrams showing the functions of the main body of the arterial pulse wave detector of FIG. 2, FIG. 7A representing a functional block diagram of one example of the invention, and FIG. 7B representing a functional block diagram of an exemplary modification of FIG. 7A;

FIG. 8 are groups of schematic time charts of signals processed by the main body of the arterial pulse wave detector of FIG. 7A, FIG. 8A representing a transmitted supersonic signal, FIG. 8B representing a received supersonic signal modulated in frequency by the Doppler effect, FIG. 8C representing an amplitude modulated signal obtained by differential amplification, and FIG. 8D representing a signal of an extracted amplitude component; and

FIG. 9 is a perspective view explanatory of a sensor portion employed by the arterial pulse wave detector of FIG. 2.

### DESCRIPTION OF THE PREFERRED EMBODIMENTS

Next, one preferred mode of the invention will be described with reference to the preferred embodiments thereof shown in the accompanying drawings.

### EXAMPLES

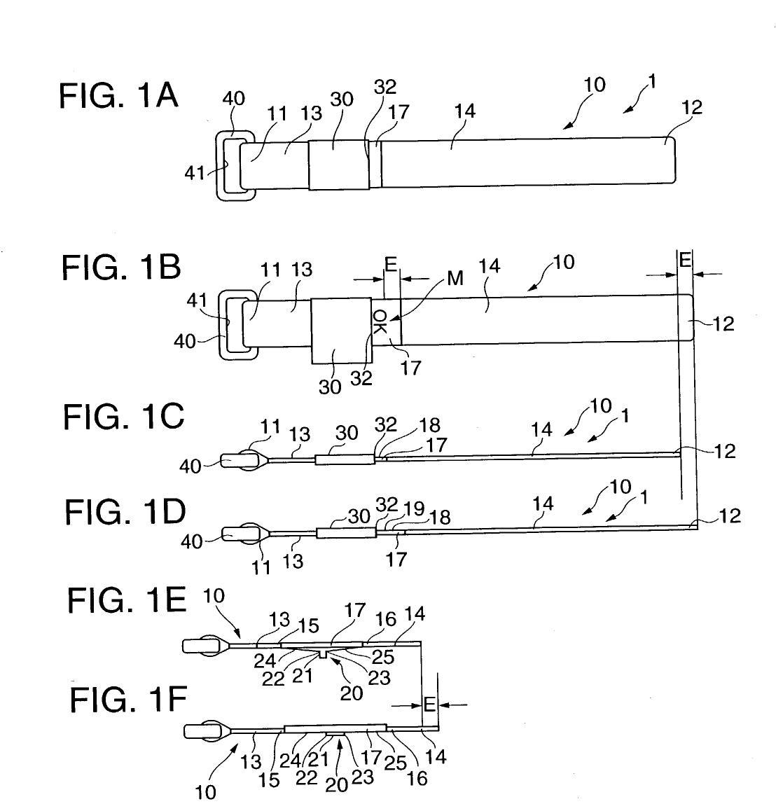

FIGS. 1 to 5 show a strap structure 1 according to one preferred embodiment of the invention and an arterial pulse wave detector 3, in an applied state, which serves as a biological information sensing device including the strap structure 1.

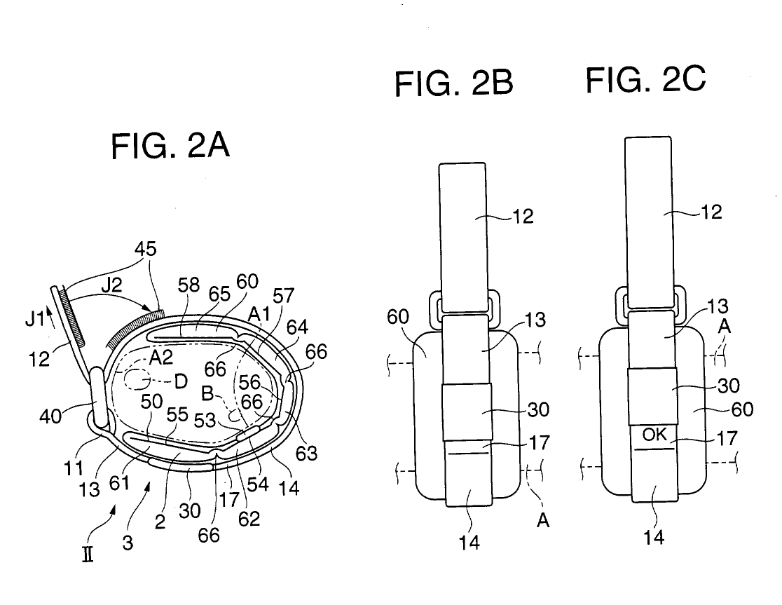

In development, as shown in FIG. 1A, the strap structure 1 includes a strap body 10 and a fastening hardware 40 attached to one end 11 of the strap body 10. The other end 12 of the strap body 10 is to be inserted through an aperture 41 of the fastening hardware 40 and is provided with strap fastening means such as hook or loop fastener 45, which cooperates with the fastening hardware 40 to fasten the strap body 10 about a wearing portion such as a wrist portion A (FIGS. 2A to 2C).

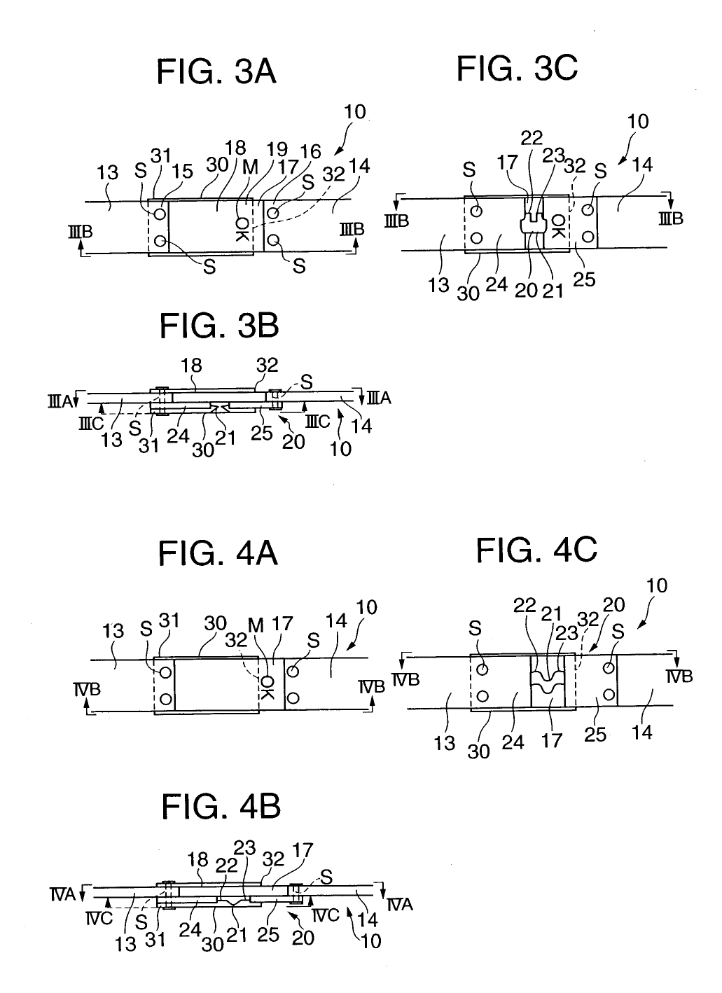

As shown in FIGS. 1A to 1F and 3A to 3C, the strap body 10 includes a first and a second strap portion 13, 14 which are flexible but substantially non-stretchable, and an elastic or stretchable strap portion 17 interposed between the first and second non-stretchable strap portions 13, 14 for connection of respective adjoining ends 15, 16 of the strap portions 13, 14.

As shown in FIGS. 1E and 1F and FIGS. 3 to 5, the strap structure 1 further includes an elongation or tension restricting means 20 interconnecting the adjoining ends 15, 16 of the non-stretchable strap portions 13, 14 in order to define an upper limit of the elongation or tension of the strap body 10. The restricting means 20 includes a non-stretchable but flexible elongation restricting strap portion 21, and non-stretchable connection pieces 24, 25 for connecting opposite ends 22, 23 of the elongation restricting strap portion 21 with the adjoining ends 15, 16 of the non-stretchable strap portions 13, 14. It is noted that the restricting means 20 may comprise the non-stretchable strap portion 21 alone.

As shown in FIGS. 1A to 1D and FIGS. 3 to 5, the strap structure 1 includes a tubular cover 30 as an indicator frame. The tubular cover 30 is attached to the strap body 10 in a manner to cover an area including the stretchable strap portion 17 and the ends 15, 16 of the non-stretchable strap portions 13, 14 of the strap body 10 that adjoin the stretchable strap portion 17. More specifically, as most apparently seen in FIGS. 3A to 3C, the tubular cover 30 comprises a tubular member of a thin rectangular section, for example, and fixed to the end 15 of the non-stretchable strap portion 13 at one end 31 thereof. A reference symbol S denotes a spot welding portion or welding portion, or fixing means such as a shaft.

On an outside surface 18 of the stretchable strap portion 17, an indicating mark M is attached to a region 19 close to the end 16 of the non-stretchable strap portion 14 in order to indicate that the amount of tension or elongation has reached a predetermined level. In the example shown in FIGS. 1 to 5, the mark comprises the characters of “OK”.

In a state where the strap body 10 of the strap structure 1 is free from tension or subjected to a minor tension, the mark M at the region near the end of the surface 18 of the stretchable strap portion 17 does not reach an end 32 of the tubular portion 30, staying within the tubular portion 30 which blocks the mark M from view, as shown in FIGS. 1A and 1C and FIGS. 3A to 3C. Therefore, a user fastening the strap structure 1 to place can visually recognize that the strap body 10 of the strap structure 1 is yet to be placed in a properly elongated or tensed state (see, for example, FIG. 1A or 3A).

When, on the other hand, a proper magnitude of tension is applied to the strap body 10 of the strap structure 1 so as to achieve an optimum amount E of elongation of the stretchable strap portion 17 of the strap body 10 of the strap structure 1, the mark M on the region 19 near the end of the surface 18 of the stretchable strap portion 17 projects beyond the end 32 of the tubular portion 30 (FIGS. 1B and 4A). As a result, the user can visually recognize that the strap body 10 of the strap structure 1 is elongated by the optimum magnitude of tension to be placed into an optimum elongation (FIGS. 1B, 2C and 4A to 4C). It is noted that the optimum amount of elongation E does not necessarily mean a strict value but only need be within a desired range based on the purpose.

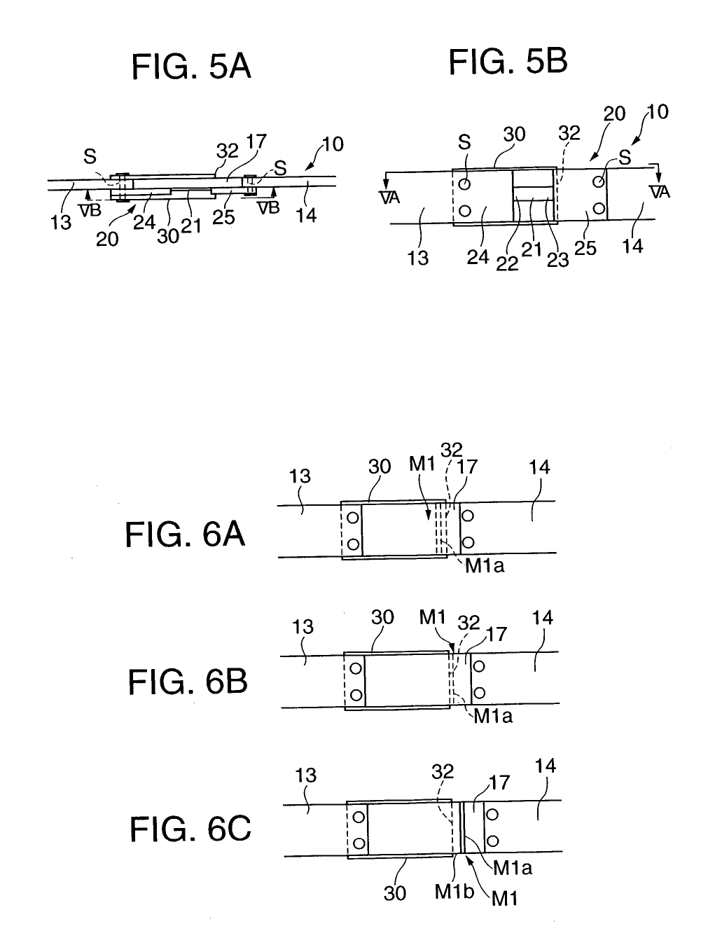

If, on the other hand, the tension on the strap body 10 of the strap structure 1 exceeds the optimum level, the stretchable strap portion 17 is stretched excessively. In an extreme case, the elongation reaches the upper limit. As a result, the non-stretchable, flexible elongation restricting strap portion 21 is stretched in maximum to become tense (FIGS. 1F, 5A and 5B). This permits the stretchable strap portion 17 to be maintained in a length defined by the elongation restricting strap portion 21 (FIGS. 5A and 5B). Hence, the stretchable strap portion 17 is prevented from being excessively elongated by an excessive tension. Thus, a fast deterioration of the strap portion 17 due to the excessive tension can be avoided.

As shown in FIGS. 2A to 2C, for example, the strap structure 1 may be assembled with a main body 2 of an arterial pulse wave detector as the main body of the biological information sensing device so that the main body 2 of the detector may be applied to the wrist A. That is, the main body 2 in combination with the strap structure 1 form the arterial pulse wave detector 3. The main body 2 of the arterial pulse wave detector may be fastened to the wrist A as secured to the non-stretchable strap portion 14 or 13 of the strap body 10 of the strap structure 1 by means of engagement means such as hook and loop fasteners.

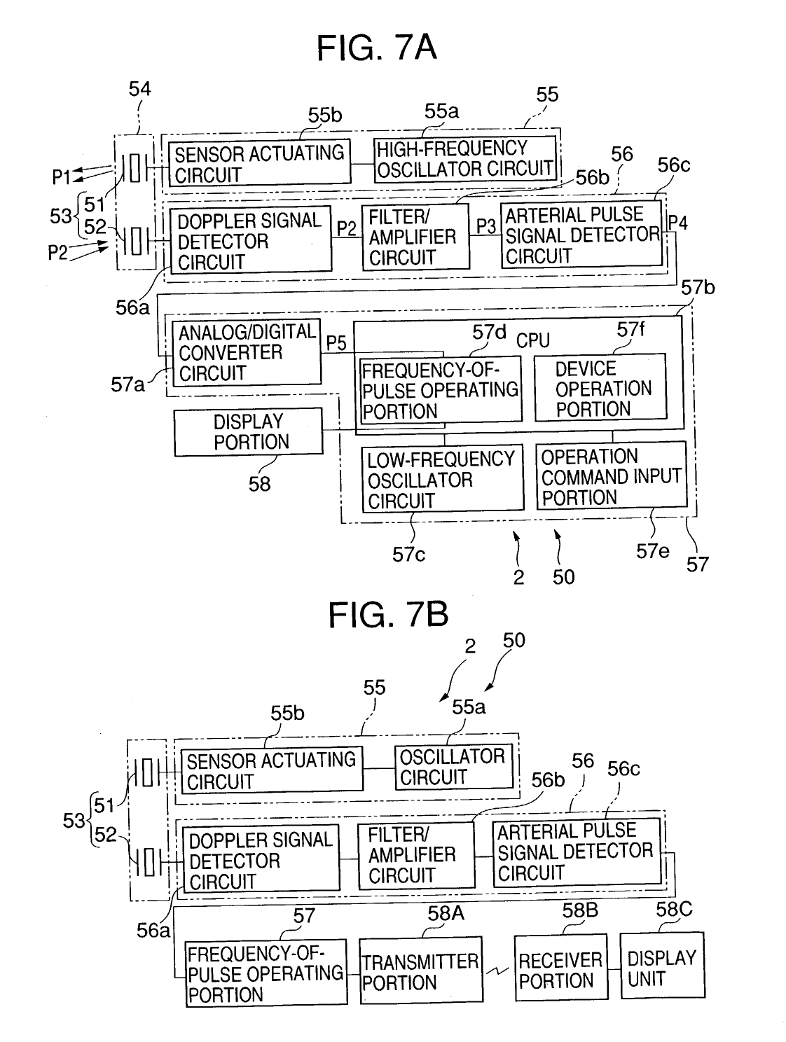

The main body 2 of the arterial pulse wave detector comprises a circuit portion 50 and a support portion 60 in the form of a flexible strap for supporting the circuit portion 50. As shown in FIG. 7A for example, the circuit portion 50 of the body 2 of the detector includes an arterial pulse wave sensor portion 54 including a sensor body 53 comprising a supersonic transmitter 51 and a supersonic receiver 52 and besides, an oscillating/actuating portion 55 for the supersonic transmitter 51 of the sensor body 53, an arterial pulse wave receiver portion 56 for extracting an analog arterial pulse signal from a supersonic signal received by the supersonic receiver 52 of the sensor body 53, a digital signal processing portion 57 for converting the arterial pulse signal extracted by the arterial pulse wave receiving portion 56 into a digital signal and processing the resultant digital signal, and a display portion 58 for display of the results of the signal processing done by the digital signal processing portion 57. These components of the circuit portion are arranged in blocks.

The circuit components 55, 54, 56, 57 and 58 constituting the circuit portion 50 of the main body 2 of the detector each comprise, for example, a printed wiring board such as formed of a resin or ceramic, or a rigid or flexible circuit board such as an integrated circuit board; and a circuit device incorporated in the circuit board. The circuit components 55, 54, 56 and 57 are each connected with the respective adjoining circuit component 54, 56, 57 or 58 via a flexible cable.

The flexible strap-like support portion 60 of the main body 2 of the arterial pulse wave detector is formed of a flexible strap material such as a urethane resin material, for example, and includes strap forming bases 61, 62, 63, 64 and 65 for supporting the respective circuit components 55, 54, 56, 57 and 58, and interconnection portions 66 for interconnecting the strap forming bases 61 to 65. The strap forming bases 61, 62, 63, 64 and 65 of the flexible strap-like support portion 60 have the corresponding circuit components 55, 54, 56, 57 and 58 mounted thereon or embedded therein.

As shown in FIG. 9 for example, the sensor portion 54 comprises a common substrate 53a incorporating therein the sensor body 53 including the supersonic transmitter 51 and the supersonic receiver 52 individually including a piezoelectric device.

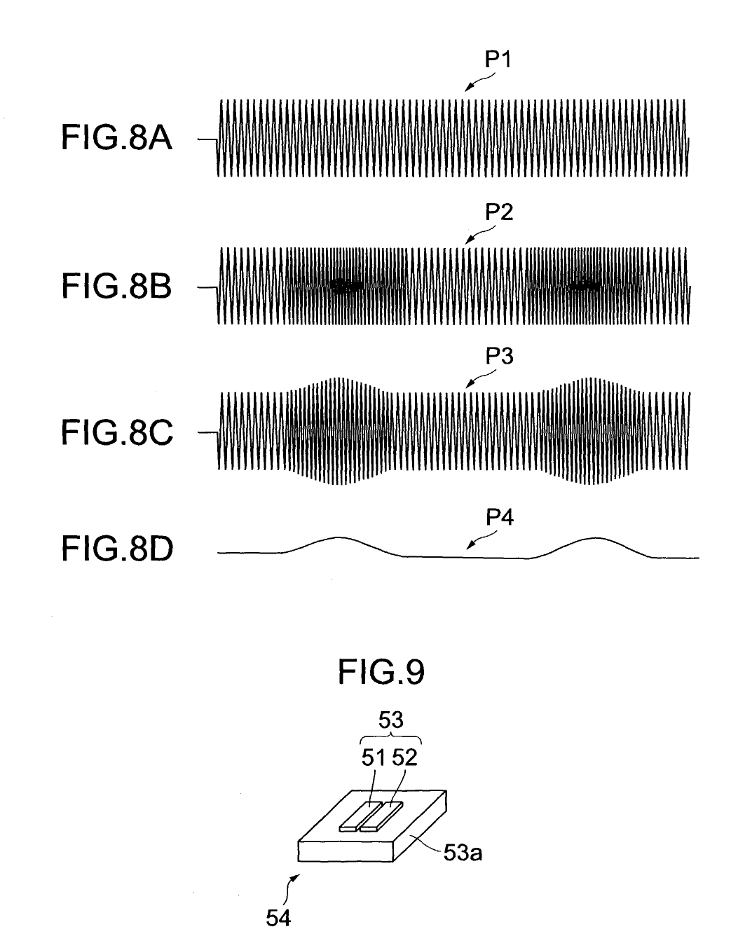

In the main body 2 of the arterial pulse wave detector, the supersonic transmitter 51 of the sensor body 53 is actuated to transmit a supersonic signal P1 under the control of the oscillating/actuating circuit portion 55 including a high-frequency oscillator circuit 55a and a sensor actuating circuit 55b, as shown in FIG. 7A, while the signal P1 is reflected as impinging upon blood components, such as blood cells or the like, in blood flowing through the radial artery B. The supersonic signal emitted from the supersonic transmitter 51 is typically the signal P1 practically having a constant frequency and amplitude, as shown in FIG. 8A. A supersonic signal P2 reflected by the blood components in blood as a pulsing stream through the radial artery B and received by the supersonic receiver 52 is modulated in frequency due to the Doppler effect associated with the pulse of the blood components as the reflector of the transmitted supersonic signal P1. Hence, the signal P2 assumes a form as shown in FIG. 8B, for example.

The arterial pulse wave receiving portion 56 for extracting an analog arterial pulse signal P4 from the supersonic signal P2 received by the supersonic receiver 52 of the sensor body 53 includes, for example, a doppler signal detector circuit 56a, a filter/amplifier circuit 56b and an arterial pulse signal detector circuit 56c, as shown in FIG. 7A. An output from the doppler signal detector circuit 56a is, for example, an electrical signal of a similar wave form P2 to that of the received supersonic signal P2. The filter/amplifier circuit 56b amplifies an amount of variation of the doppler signal P2˜sin{(ω+Δω)t} using the original transmission signal P1˜sin(ω·t) as a reference or a reference signal, so as to extract a differential amplification signal P3 as shown in FIG. 8C. It is noted here that ω denotes an angular frequency of the supersonic signal P1, and that Δω=Δω(t) denotes a modulated angular frequency dependent upon time t due to the Doppler effect. In the arterial pulse wave receiving portion 56, the arterial pulse signal detector circuit 56c extracts, as the arterial pulse signal P4, an amplitude modulated component from the differential amplification signal P3.

Although FIG. 8 show the arterial pulse wave P4 quite in a simple wave form, the arterial pulse wave P4 actually presents much more complicated time-dependent wave form than that of FIG. 8D. Particularly in a state where the cardiopulmonary circulatory system is overtaxed during or after exercise, the arterial pulse wave P4 assumes a much more complicated and irregular wave form containing a wide range of high frequency components.

In the case of the main body 2 of the arterial pulse wave detector shown in FIG. 7A, the digital signal processing portion 57 includes an analog/digital (A/D) converter circuit 57a for converting the analog signal P4 indicative of the arterial pulse wave into a digital signal P5 indicative of the arterial pulse wave, a central processing unit (CPU) 57b for receiving the digital arterial pulse signal P5, and a low-frequency oscillator circuit 57c for supplying the CPU 57b with a reference signal for processing. In this case, the CPU 57b includes a memory for storing a frequency-of-pulse operation program and a microprocessor for executing the program in order to forma frequency-of-pulse operating portion 57d for operating the frequency of pulse based on the digital arterial pulse signal P5 with reference to the low-frequency signal from the low-frequency oscillator circuit 57c. Typically, the CPU forms a digital signal processor (DSP) wherein a part of the frequency-of-pulse operation program including a fast Fourier transformation (FFT) process is incorporated in a digital signal processor circuit. It is noted that the CPU 57b further includes a device operation portion 57f for receiving an operation command from an operation command input portion 57e such as a push-button switch. According to FIG. 7A, the display portion 58 comprises a display unit for displaying the operation result or frequency of pulse Q determined by the frequency-of-pulse operating portion 57d of the CPU 57b.

In a case where the main body 2 of the arterial pulse wave detector includes the display portion 58, the strap forming base 65 including the display portion 58 may be provided with the display portion at place near one end thereof with respect to a width-wise direction ω thereof or at a central portion thereof with respect to the width-wise direction thereof. In the former case, a region of the strap body 10, that overlaps the strap forming base 65, is formed in a smaller width than that of the strap forming base 65 in order to provide the view of the display on the display portion 58. Alternatively, the strap body 10 may be formed in a width equal to or greater than that of the strap forming base 65 and a side edge portion of the strap forming base 65, that is overlapped by the display portion 58, may be formed of a transparent material. In the latter case, the strap body 10 includes an opening at its region overlapped by the display portion 58 of the strap forming base 58 in order to provide the view of the display on the display portion 58 disposed centrally with respect to the width-wise direction of the strap body. Of course, the opening may be formed of a transparent material.

Needless to say, the CPU 57b may perform other operations during spare-time when the frequency of pulse Q is not operated or at an interval between the operations of the frequency of pulse Q. One example of the other operations include a time counting operation as a clock. Specifically, the CPU 57b is, for example, capable of performing the time counting operation as a timer and hence, the display portion 58 is also capable of functioning as a display of a digital clock.

The arterial pulse wave detector 3 of the above construction may be worn on the wrist A as follows. As shown in FIG. 2A, the main body 2 of the arterial pulse wave detector 3 is placed around the wrist A in a manner to bring the sensor body 53 of the sensor portion 54 into abutment against a wrist surface area A1 near the radial artery B of the wrist A. With the fastening hardware 40 of the strap structure 1 positioned at a wrist bump area A2 near the cubitus D, the end 12 of the strap body 10 is inserted through the aperture 41 of the fastening hardware 40 and pulled along directions J1 and J2. Then, the end portion 12 is fixed by means of hook and loop fasteners 45, 45 under conditions shown in FIG. 2C corresponding to FIGS. 4A to 4C, wherein the indicator mark M indicative of a fastened or tense state is just projected from the tubular portion 30 to indicate the optimum tension or elongation.

With the arterial pulse wave detector 3 wrapped around the wrist A in this manner, the sensor body 53 of the main body 2 of the detector 3 can be maintained in intimate contact with the measurement area A1 of the wrist A at the optimum fastening force or pressure applied by the strap structure 1 in the optimum tension or elongation and hence, the precise measurements of the arterial pulses can be taken. Furthermore, the main body 2 of the detector 3 substantially has a uniform mass distribution along the longitudinal direction thereof. Therefore, even when the wrist A is subjected to an accelerative motion due to exercise or the like, such a great inertia force as to bring the arterial pulse wave detector 3 into mono-directional rotation about the wrist A will not occur actually. Accordingly, the sensor body 53 is less susceptible to the variations of the pressure thereon or the displacement thereof, thus resulting in a decreased fear of the invasion of noises during the signal processings.

In the main body 2 of the arterial pulse wave detector, the direct display of the frequency of pulse Q on the display portion 58 as shown in FIG. 7A may be replaced by the following arrangement. As shown in FIG. 7B, a transmitter portion 58A including an antenna or coil is adapted to transmit data on the frequency of pulse Q, obtained by the frequency-of-pulse operating portion 57, in the form of an electromagnetic signal R such as of an electromagnetic wave or variable magnetic field, whereas a separate receiver portion 58B is provided for receiving the electromagnetic signal R, from which the frequency of pulse Q is extracted to be displayed on a display unit 58C.

The foregoing description illustrates the example where the elongation or tension indicating mark M comprises the characters “OK”. However, as shown in FIG. 6, a mark M1 comprising lines of dark and pale colors or a color pattern is also usable. Referring to FIGS. 6A to 6C, a black bar (dark color) portion M1a is used as reference. When the bar portion M1a is hidden under the tubular portion 30 (FIG. 6A), it indicates that a tension is below the optimum level. When projected far beyond the tubular portion 30 to expose a white (pale color) region M1b in juxtaposition with the bar portion M1a (FIG. 6C), the bar portion M1a indicates a tension much greater than the optimum level. When properly exposed from the tubular portion 30 (FIG. 6B), the bar portion M1a indicates a tension at the optimum level.

What is claimed is:

1. A strap structure comprising:
a strap body for being wrapped around a neck-like portion; wherein the strap body comprises an elastic strap-like portion elastically elongated according to a tension applied to the strap body, and an elongation indicator for indicating an elongation of the elastic strap-like portion.

2. A strap structure as claimed in claim 1, wherein the elongation indicator includes an indicator frame having one end thereof attached to a non-elastic strap-like portion connected with one end of the elastic strap-like portion and the other end thereof extended along an extension direction of the elastic strap-like portion; and an elongation indicating mark attached to a portion of the strap body that corresponds to the extended end of the indicator frame and is designed to be exposed from the indicator frame.

3. A strap structure as claimed in claim 1, further comprising an elongation restricting member for defining an upper limit of the elongation of the elastic strap-like portion.

4. A strap structure comprising:
a strap body for being wrapped around a neck-like portion; wherein the strap body comprises an elastic strap-like portion elastically elongated according to a tension applied to the strap body, and a tension indicator for indicating a tension applied to the elastic strap-like portion.

5. A strap structure as claimed in claim 4, wherein the tension indicator includes an indicator frame having one end thereof attached to a non-elastic strap-like portion connected with one end of the elastic strap-like portion and the other end thereof extended along an extension direction of the elastic strap-like portion; and a tension indicating mark attached to a portion of the strap body that corresponds to the extended end of the indicator frame and is designed to be exposed from the indicator frame.

6. A strap structure as claimed in claim 4, further comprising a tension restricting member for defining an upper limit of the tension applied to the elastic strap-like portion.

7. A strap structure as claimed in claim 1, further comprising a fastening hardware provided at one end of the strap body and including an aperture in which the other end of the strap body is inserted; and fixing member for fixing the other end portion of the strap body inserted through the aperture of the fastening hardware in a manner to allow for the definition of an optional length of the inserted other end portion.

8. A strap structure as claimed in claim 4, further comprising a fastening hardware provided at one end of the strap body and including an aperture in which the other end of the strap body is inserted; and fixing member for fixing the other end portion of the strap body inserted through the aperture of the fastening hardware in a manner to allow for the definition of an optional length of the inserted other end portion.

9. A biological information sensing device comprising:
a main body for being worn on a neck-like portion, inclusive of a predetermined measurement area, of a living organism as holding a sensor portion thereof in intimate contact with the measurement area for sensing biological information of the living organism;
wherein the main body including the sensor portion and a flexible strap-like sensor holder for holding the sensor portion; and
a strap structure comprising a strap body for being wrapped around a neck-like portion; wherein the strap body comprises an elastic strap-like portion elastically elongated according to a tension applied to the strap body, and an elongation indicator for indicating an elongation of the elastic strap-like portion,
wherein the strap structure serves to fasten the main body of the biological information sensing device to the neck-like portion of the living organism.

10. A biological information sensing device comprising:
a main body for being worn on a neck-like portion, inclusive of a predetermined measurement area, of a living organism as holding a sensor portion thereof in intimate contact with the measurement area for sensing biological information of the living organism;
wherein the main body including the sensor portion and a flexible strap-like sensor holder for holding the sensor portion; and
a strap structure comprising a strap body for being wrapped around a neck-like portion; wherein the strap body comprises an elastic strap-like portion elastically elongated according to a tension applied to the strap body, and a tension indicator for indicating a tension applied to the elastic strap-like portion,
wherein the strap structure serves to fasten the main body of the biological information sensing device to the neck-like portion of the living organism.

11. A biological information sensing device as claimed in claim 9, wherein the sensor portion is a sensor for sensing information on blood flowing through the radial artery.

12. A biological information sensing device as claimed in claim 10, wherein the sensor portion is a sensor for sensing information on blood flowing through the radial artery.
